(19) United States
(12) Patent Application Publication
(10) Pub. No.: US 2023/0255740 A1
(43) Pub. Date: Aug. 17, 2023

(54) THREE-DIMENSIONAL MESH ARRANGEMENTS FOR USE IN TISSUE ENGINEERING AND SURGICAL APPLICATIONS

(71) Applicant: SURGICAL INNOVATION ASSOCIATES, INC., Chicago, IL (US)

(72) Inventors: Stephanie Lietz, Chicago, IL (US); Todd Cruikshank, Chicago, IL (US); Hilton Becker, Boca Raton, FL (US); Alexei Mlodinow, Chicago, IL (US); Josh Vose, Chicago, IL (US)

(73) Assignee: SURGICAL INNOVATION ASSOCIATES, INC., Chicago, IL (US)

(21) Appl. No.: 18/168,235

(22) Filed: Feb. 13, 2023

Related U.S. Application Data
(63) Continuation-in-part of application No. 17/684,043, filed on Mar. 1, 2022.
(60) Provisional application No. 63/309,841, filed on Feb. 14, 2022.

(51) Int. Cl. A61F 2/00

(52) U.S. Cl. CPC A61F 2/0063; A61F 2002/0068

(57) ABSTRACT

An arrangement includes: a plurality of mesh tubes arranged in parallel and extending along an axial direction, each mesh tube of the plurality of mesh tubes having a mesh tube length in the axial direction; and a first mesh sheet having a first surface. The plurality of mesh tubes is disposed on the first surface. The arrangement provides structural support to surrounding tissues upon placement of the arrangement into a human body.

### CROSS-REFERENCE TO RELATED APPLICATIONS

This patent application claims the benefit of U.S. Provisional Patent Application No. 63/309,841, filed Feb. 14, 2022, and is a continuation-in-part of U.S. application Ser. No. 17/684,043, filed Mar. 1, 2022, the entire disclosures of which are incorporated herein by reference.

### FIELD

The present disclosure relates to three-dimensional mesh (e.g., surgical mesh) arrangements for a variety of applications, including surgical implantation and tissue engineering.

### BACKGROUND

Absorbable mesh is beneficial for use in surgical procedures where tissue support and tissue induction are desired. Potential benefits to certain types of absorbable mesh include the characteristics of being lightweight and flexible, as well as the ability to dissolve into the patient's own tissue over time. The density of an absorbable mesh also allows for tissue to grow through and around the mesh as the mesh is absorbed, so that the support offered by the mesh can eventually be replaced with the body's own tissues. Different absorbable meshes can be absorbed into the body over different timeframes. Moreover, there are also permanent meshes that do not dissolve into the body. In general, such meshes are currently only available in flat sheets of various sizes and shapes.

However, in some procedures, such as breast reconstruction, enhanced structural support with increased volume and space may be desired or required while maintaining the benefits of absorbable mesh as described above. Unfortunately, conventional means of adding structural support to an absorbable mesh have required the thickening of the mesh fibers, resulting in a loss of flexibility. These methods also result in an increased mesh density, which can lead to an enhanced risk of seroma formation, infection, and/or lack of tissue support upon absorption of the mesh into the patient's body.

A more recent development is the incorporation of bioactive substances with the mesh. For example, platelet-rich plasma (obtained from the patient's blood), fat obtained from the patient, and/or other biological or synthetic agents (e.g., hyaluronic acid) can be incorporated into the mesh, thereby forming a “bio-mesh.” It is difficult, however, to apply and retain these biological or synthetic agents on flat mesh sheets, which limits the likelihood of success when using such flat mesh sheets in a surgical procedure.

### SUMMARY

In an embodiment, the present invention provides an arrangement, comprising: a plurality of mesh tubes arranged in parallel and extending along an axial direction, each mesh tube of the plurality of mesh tubes having a mesh tube length in the axial direction; and a first mesh sheet having a first surface, wherein the plurality of mesh tubes is disposed on the first surface.

### BRIEF DESCRIPTION OF THE DRAWINGS

FIG. 1A shows a flat mesh sheet;

FIG. 1B shows a close-up view of the flat mesh sheet of FIG. 1A;

FIG. 2A shows an embodiment of a three-dimensional mesh arrangement according to the present disclosure;

FIG. 2B shows an exemplary cross-section of the three-dimensional mesh arrangement of FIG. 2A;

FIG. 2C shows an another exemplary cross-section of the three-dimensional mesh arrangement of FIG. 2A,

FIG. 2D shows a top view of the three-dimensional mesh arrangement of FIG. 2A:

FIG. 2E shows two instances of the three-dimensional mesh arrangement of 2A disposed in a patient's body;

FIG. 2F shows a three-dimensional mesh arrangement comprising the three-dimensional mesh arrangement of FIG. 2B stacked with two instances of the flat mesh sheet of FIGS. 1A-1B;

FIG. 2G shows a three-dimensional mesh arrangement comprising the three-dimensional mesh arrangement of FIG. 2C stacked with two instances of the flat mesh sheet of FIGS. 1A-1B;

FIG. 3A shows a top view of another embodiment of a three-dimensional mesh arrangement according to the present disclosure;

FIG. 3B shows a front view of the three-dimensional mesh arrangement of FIG. 3A;

FIG. 3C shows a three-dimensional mesh arrangement comprising the three-dimensional mesh arrangement of FIGS. 3A-3B stacked with the flat mesh sheet of FIGS. 1A-1B;

FIG. 31) shows a three-dimensional mesh arrangement comprising a stack of three instances of the three-dimensional mesh arrangement of FIGS. 3A-3B;

FIG. 4 shows yet another embodiment of a three-dimensional mesh arrangement according to the present disclosure;

FIG. 5A shows still another embodiment of a three-dimensional mesh arrangement according to the present disclosure, the three-dimensional mesh arrangement containing a biological or synthetic agent;

FIG. 5B shows a top view of the three-dimensional mesh arrangement of FIG. 5A,

FIG. 6A shows a further embodiment of a three-dimensional mesh arrangement according to the present disclosure;

FIG. 6B shows two instances of the three-dimensional mesh arrangement of FIG. 6A; and

FIG. 6C the two instances of the three-dimensional mesh arrangements of FIG. 6B coated in a biological or synthetic agent.

### DETAILED DESCRIPTION

As used herein, the terms “mesh,” “mesh sheet,” and “scaffold” are used interchangeably and are not intended to have a different meaning, unless clearly indicated by context.

As used herein, “absorbable” and “resorbable” are used interchangeably and are not intended to have a different meaning, unless clearly indicated by context. “Absorbable” and “resorbable” mean that a mesh breaks down over a specified period of time (specified elsewhere herein) under physiological conditions (i.e., without the aid of external assistance, such as sonication, drugs, palpation, or surgery), There are different degrees of absorbability or resorbability. For example, a mesh that is “fully” resorbable within six months would mean that the mesh is no longer present at the site of implantation by the six-month mark.

As used herein, a “wave shape,” “corrugations,” and “pleats” in reference to a mesh sheet are used interchangeably and mean a shape having a series of peaks and valleys in repeating fashion. A wave shape can have a smooth curvature between the peaks and valleys, a curvature that is not smooth, such as a square shape or a triangular shape, or a combination of both smooth curvature for some peaks/valleys and not smooth for other peaks/valleys. For example, a wave shape can include a combination of smooth curvature in one portion of a mesh sheet and a boxy shape in another portion of the mesh sheet.

As used herein, the term “volume” is generally meant to include both open space (e.g., voids) and, if present, any structures (i.e., material such as a mesh sheet or connecting threads) present in a given volume. For example, in aspects when an arrangement has a first layer and a second layer to form a volume therebetween, the volume generally includes any open space (e.g., voids), as well as any structures present in the volume between the first and second layers.

As used herein, “mesh porosity” in reference to the mesh means the void fraction within the volume of the mesh. Mesh porosity is calculated according to the following equation:

$$P = 100 [ \frac{1 - M}{1000} \times h \times ρ ]$$

in which P is mesh porosity, M is the mass per unit area (g/m²) of the mesh, h is the thickness (mm) of the mesh in the direction perpendicular to the mesh face, and p is the relative density (g/cm³) of the material making up the mesh (e.g., the thread, polymer, fabric, etc.).

As used herein, the terms “memory” and “memory characteristics” in reference to a mesh sheet are meant to indicate that a mesh sheet can be formed or manipulated into a specific shape and, due to the material properties of the mesh sheet, will generally remain in that shape. This property of the mesh sheets is particularly useful in forming the three-dimensional mesh arrangements described herein. The memory characteristic is not so strong as to make the mesh sheets rigid.

As used herein, “channel” means the shortest direct pathway between one face of a mesh and the opposite face of the mesh that allows fluids, cells, blood vessels, and/or tissues entrance to and egress from the interior volume of the mesh. Channels may be uniform throughout their length, or may not be uniform throughout their length (e.g., narrowing or widening along their length, or having a different shape than the channel openings) depending, for example, on the structure of a cross-layer stitching. Channels connect the channel openings on the two faces of the mesh. Generally, the channels are the largest pathways within the mesh connecting the two faces of a mesh.

As used herein, a “channel opening” is the opening in the two-dimensional face of a mesh that is the entrance to a channel. A channel opening can have any suitable shape, such as a honeycomb shape or other shape, as characterized by viewing the mesh perpendicular to the face it is present in. Generally, channel openings are the largest openings in the two-dimensional face of a mesh, and as such channel openings are to be contrasted with other generally smaller openings (e.g., “minor pores”) in a face of the mesh that may exist due to, for example, gaps inherent to the nature of the intertwining of the material that makes up the mesh (e.g., threads or yarn). As the mesh gets thinner, the channel opening and the channel become synonymous, as in a two-dimensional mesh.

As used herein, “internal passageway” means a pathway within the plane of the mesh (i.e., between the two faces of the mesh) that allows fluids, cells, blood vessels, and/or tissues to move within the internal volume of the mesh. Internal passageways generally fluidically connect two or more channels.

As used herein, “macroporous structure” means a system of one or more of channels, channel openings, internal passageways, and minor pores present in a mesh. Typically, a mesh comprises each of openings, channel openings, channels, internal passageways, and minor pores, and thus the macroporous structure includes all of these features. However, in some aspects, certain features, such as the minor pores, may not be present, and therefore the macroporous structure in such a case would include only the channel openings, channels, and internal passageways. The macroporous structure, in some aspects, provides the mesh with various properties, such as a low density, ability to absorb fluids, structural integrity, or any combination thereof. Generally, the plurality of channel openings on a face of the mesh are joined through the interior of the mesh via channels. In some aspects, the channels are joined within the interior (e.g., volume) of the mesh by internal passageways. In some aspects, the minor pores also provide a route into and out of the interior, or at least provide a location for fluids, compounds, and/or cells to adhere to the mesh.

To overcome problems associated with the prior art, the present application describes various mesh arrangements that are particularly well-suited for, among other things, providing support to adjacent tissues within a patient's body. To form these arrangements, each mesh sheet may be flexible and elastic so the resulting arrangements can be draped and adjusted as needed. As such, each mesh sheet can be molded an expanded by the surgeon implanting the mesh sheets into the patient's body.

The resulting arrangements may be three-dimensional so as to create volume within a cavity in the patient's body in which each arrangement is placed. To assist in imparting three-dimensionality to the arrangements, each mesh sheet has memory characteristics that allow the mesh sheet to maintain its shape after manipulation (e.g., by a surgeon preparing to implant one or more mesh sheets into the patient's body). By using mesh sheets to create three-dimensional mesh arrangements, the mesh sheets are better able to retain biological and synthetic agents applied thereto, which increases the likelihood that the arrangements will be accepted within the patient's body and provide the required tissue support. The three-dimensional nature of the arrangements also facilitates incorporation of biological and synthetic agents thereon.

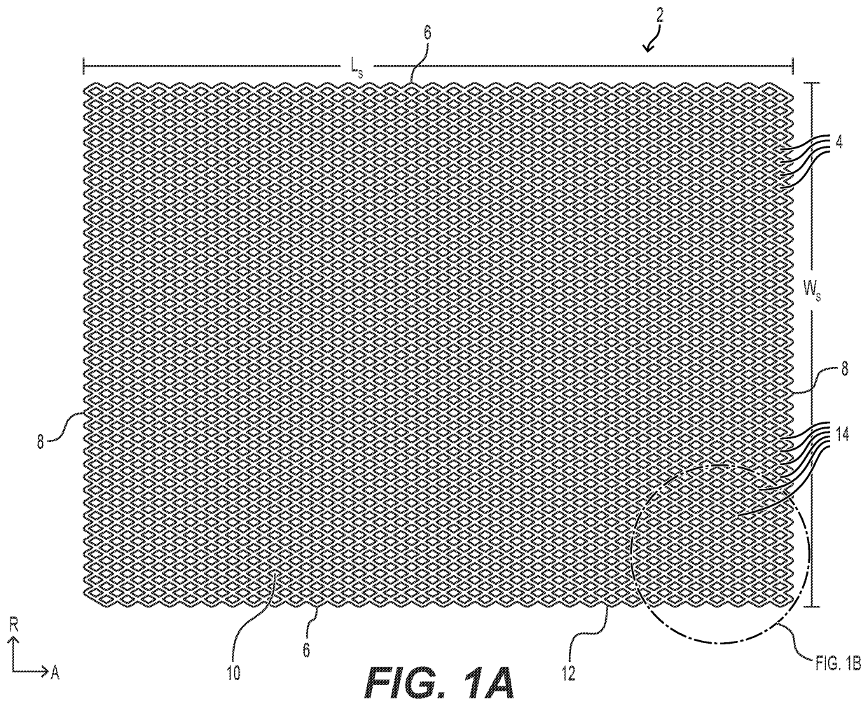

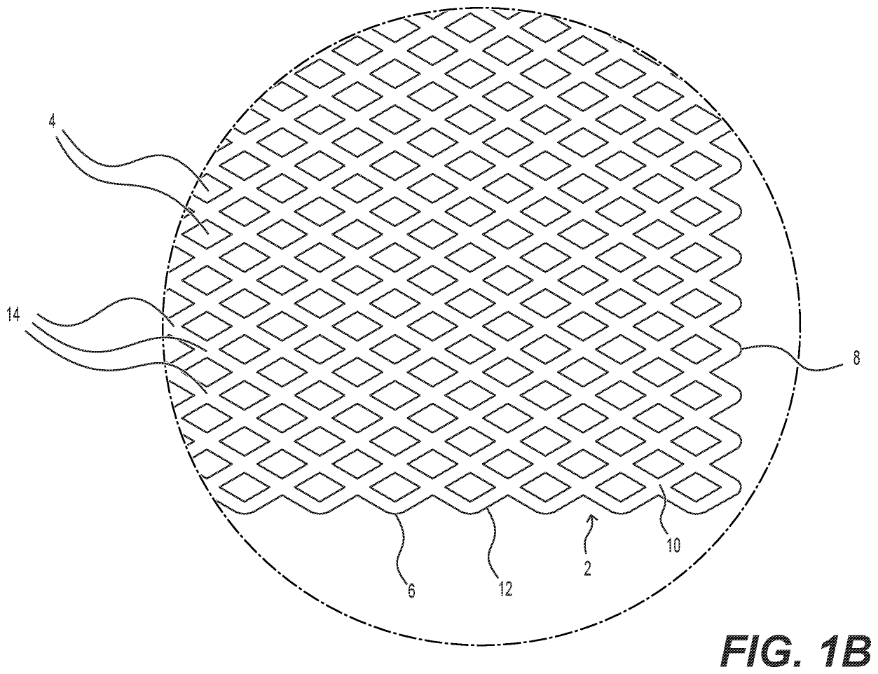

FIG. 1 of the present application shows an example of a mesh sheet 2 described in U.S. application Ser. No. 17/684,043 (“the '043 application”), to which the present application claims priority. In particular, FIG. 1A of the '043 application illustrates a mesh or scaffold 100 that can be used, for example, as a reinforced absorbable surgical mesh, including as mesh sheet 2 of the present application. Consequently, when the present application refers to mesh sheet 2, it should be understood that mesh sheet 2 is one in the same as mesh or scaffold 100 described in the '043 application. When the present application refers to characteristics of mesh sheet 2, it should also be understood that mesh sheet 2 can have any or all the characteristics of mesh or scaffold 100 described in the '043 application. In any event, however, the three-dimensional mesh arrangements described herein can use a variety of meshes, including meshes other than mesh or scaffold 100 described in the '043 application, such that the three-dimensional mesh arrangements of the present application should not be limited to using only mesh or scaffold 100 described in the '043 application.

FIG. 1B of the '043 application is a close-up view of a portion of FIG. 1A of the '043 application. The mesh or scaffold 100 therein comprises a plurality of channel openings 102 having a hexagon shape, providing the mesh or scaffold 100 with an overall honeycomb-shaped structure. The channel openings 102 on each face or surface are connected by channels 112 that traverse the interior of the mesh. FIG. 4B of the '043 application also shows channel openings 400, channels 402, and minor pores 404.

In some aspects, the macroporous structure of mesh sheet 2, including channels in combination with internal passageways and/or minor pores, is sponge-like, facilitating adsorption and/or absorption of fluids, cells, and/or other compounds. In some aspects, biological or synthetic agents can be added to mesh sheet 2 to stimulate tissue ingrowth. In some aspects, the macroporous structure of mesh sheet 2 facilitates tissue ingrowth when implanted within the patient to create the native tissue support that is desired as mesh sheet 2 adsorbs and/or absorbs fluids, cells, biological or synthetic agents, compounds, or any combination thereof. Examples of fluids, cells, biological or synthetic agents, or compounds include, for example, adipose tissue, platelet-rich plasma (PRP), hyaluronic acid, lipoaspirate stromal vascular fraction, compositions containing heterogeneous cells from the patient, regenerative or therapeutic agents (e.g., to facilitate healing, cellular in-growth, and/or cellular repair), or any combination thereof.

In some aspects, hyaluronic acid in particular is useful for promoting tissue ingrowth. For example, when hyaluronic acid (or similar fluids or compounds) are added to mesh sheet 2 as a synthetic agent, cell migration into the fluid-containing spaces can be stimulated. These cells, which can be fibroblasts, then lay down collagen, thereby facilitating cell growth. In some aspects, growth factors other than hyaluronic acid can alternatively or additionally be added to mesh sheet 2. In some aspects, a compound or fluid of interest (e.g., hyaluronic acid) can be coated onto and/or infused within mesh sheet 2 and then used as is in a desired application (e.g., surgery or tissue engineering). Alternatively, the coated/infused mesh sheet 2 can first be dried and used as-is in a desired application, such as in situations where the dried mesh sheet 2 does not detrimentally affect the desired application. However, in other aspects, the dried mesh sheet 2 is rehydrated in sterile water prior to use in a desired application. In such an aspect, the rehydration causes compounds of interest, particularly hydrophilic compounds such as hyaluronic acid, to fill the channels and other internal spaces of mesh sheet 2.

In some aspects, mesh sheet 2 is composed of a yarn. In some aspects, the yarn comprises a monofilament fiber yarn, a multifilament fiber yarn, or a combination thereof. In some aspects, the yarn comprises a polymer, a copolymer, or a combination thereof. In some aspects, the yarn is absorbable and/or resorbable. In some aspects, the yarn may be equivalent to USP 7-0, 6-0, 5-0, 4-0, 3-0, 2-0, or 1-0, or any combination thereof. Lower-diameter fibers such as 6-0 and 5-0 can be used to elicit a lower macrophage count and/or a lower overall diameter of the associated inflammatory infiltrate compared to larger-diameter fibers, and thus may be desired in certain contexts that benefit from such properties. In some aspects, such properties have positive implications for tissue ingrowth and long-term strength. On the other hand, higher caliber fibers are generally stronger, which has implications for short-term strength of mesh sheet 2 in the period after implantation, and before tissue ingrowth.

In some aspects, suitable polymers that compose mesh sheet 2 may include, for example, polydioxanone, poly-4-hydroxybutyric acid, polylactic acid, poly-glycolic acid, polycaprolactone, trimethylene carbonate, any copolymer thereof or any combination thereof. Other suitable polymers may be used as known in the art. In some aspects, the polymer anchor copolymer yarns used in manufacture of mesh sheet 2 are woven and/or warp-knitted in stitch patterns creating the channel opening shapes and/or channel shapes as described in the '043 application.

Selection of the polymer(s) used in forming mesh sheet 2 may affect the effective porosity, the effective channel diameter, and/or the effective channel opening diameter, each of which collectively contributes to the mesh porosity of mesh sheet 2. The discussion in the '043 application of the benefits of porosity and channel and/or channel opening size assumes that these channels and channel openings are not blocked by bridging granulomata or bridging scar tissue, both clinically-problematic effects of the response to a foreign body, such as mesh sheet 2. The bridging of channel openings or the clogging of channels is termed “encapsulation” rather than “incorporation,” and generally precludes neovascularization; tissue ingrowth, fluid flow, and other positive elements of the foreign body response. Effective porosity is used to describe the porosity (and channel or channel opening size) when taking into account the bridging granulomata and inflammatory infiltrate (sizes ranging from ˜80 to ˜400 microns) that will form at the periphery of foreign fibers. Polymer material, degradation byproducts, filament size, and other factors alter the effective size of the channel openings and channels in mesh sheet 2. Effective porosity, effective channel opening diameter, and effective channel diameter contribute to the mesh porosity. Individually, each of the pores can be measured by microscopy or metrology techniques.

Selection of the polymer(s) used in forming mesh sheet 2 may also allow the resulting arrangement to be at least partially resorbable or, in some aspects, fully resorbable. Mesh sheet 2 may fully degrade/resorb in situ within about 1 month, about 2 months, about 3 months, about 4 months, about 5 months, 6 months, about 7 months, about 8 months, about 9 months, about 10 months, about 11 months, about 12 months, about 13 months, about 14 months, about 15 months, about 16 months, about 17 months, or about 18 months, or any, range derived therefrom, such as 1-18 months 2-17 months, 3-16 months, 4-15 months, 5-14 months, 6-13 months, 7-12 months, 8-11 months, 9-10 months, 1-12 months, 6-18 months, 1-4 months, 4-8 months, 8-12 months, 12-16 months, or 16-1S months. In some aspects, mesh sheet 2 may fully degrade/resorb within a greater or lesser period of time as desired. In this respect, the degradation/resorption timing of any aspect is based not only upon polymer selection and fiber configuration, but also upon fiber caliber, number of layers, cross-layer stitch pattern, channel opening average diameter, and channel average diameter. In some aspects, mesh sheet 2 is not resorbable (i.e., mesh sheet 2 is permanent) as that term would be understood by those of ordinary skill in the art of implanting surgical meshes in patients.

Returning to FIGS. 1A-1B of the present application, mesh sheet 2 includes a number of openings 4 (corresponding to channel openings 102 described the '043 application) therein through which the tissue can grow as mesh sheet 2 is absorbed into the patient's body, lending support to the adjacent tissue. Mesh sheet 2 forms the basic building block of the three-dimensional mesh arrangements described herein.

As shown, mesh sheet 2 has a generally rectangular shape. However, mesh sheet 2 can also come in other shapes or be cut into any desired shape. In the embodiment shown shown, mesh sheet 2 has a mesh sheet length L_{S} that extends in axial direction A and a mesh sheet width W_{S} that extends in radial direction R perpendicular to axial direction A. Mesh sheet length L_{S} is greater than mesh sheet width W_{S}. In this manner, mesh sheet 2 has two lengthwise edges 6 that are parallel to each other and two widthwise edges S that are parallel to each other. Given its generally two-dimensional shape, mesh sheet 2 also has a first face or surface 10 on one side of mesh sheet 2 and a second face or surface 12 on the opposite side of mesh sheet 2, each surface 10, 12 extending in axial direction A and radial direction R.

Openings 4 in mesh sheet 2 can be large enough that a suture or connecting thread can be passed through each opening 4, as discussed in more detail below, to secure one portion of mesh sheet 2 to another portion of mesh sheet 2, or to secure one portion of mesh sheet 2 to a portion of another mesh sheet 2. Between any two adjacent openings 4 is a node 14, around which a connecting thread or suture can be wrapped. Depending on the configuration of openings 4, an opening 4 in the middle of mesh sheet 2 can be surrounded by a plurality of nodes 14. In the example shown in FIG. 1A, an opening 4 in mesh sheet 2 is surrounded by at least eight nodes 14, one for each circumferentially adjacent opening 4. The mesh material at any given node 14 can be glued or melted to connect that node 14 with one or more other nodes 14, whether of the same mesh sheet 2 or a different mesh sheet 2. FIG. 1B shows a close-up view of mesh sheet 2 of FIG. 1A.

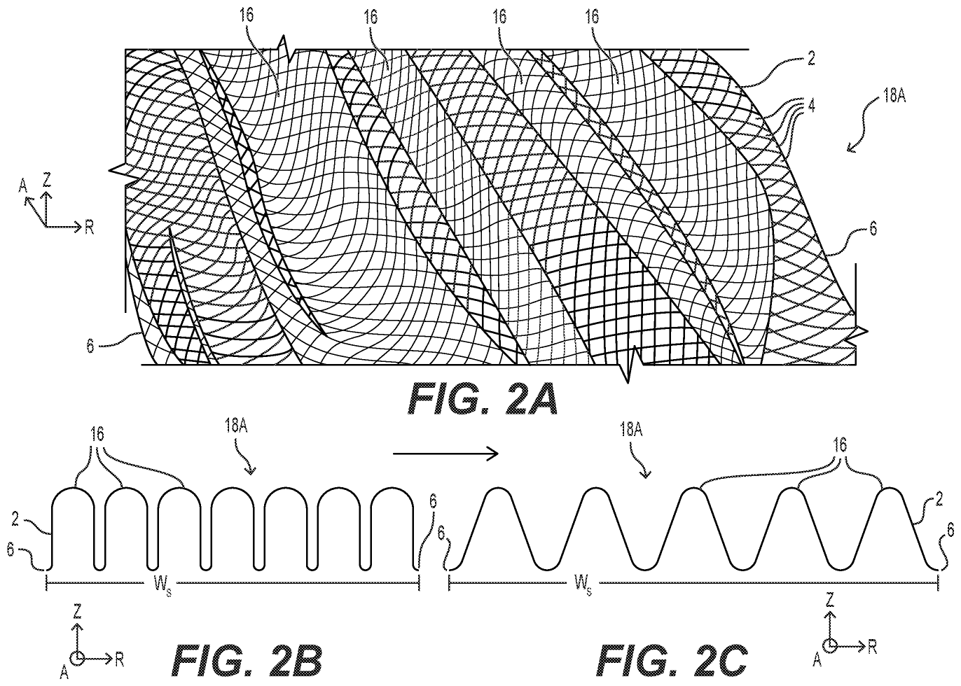

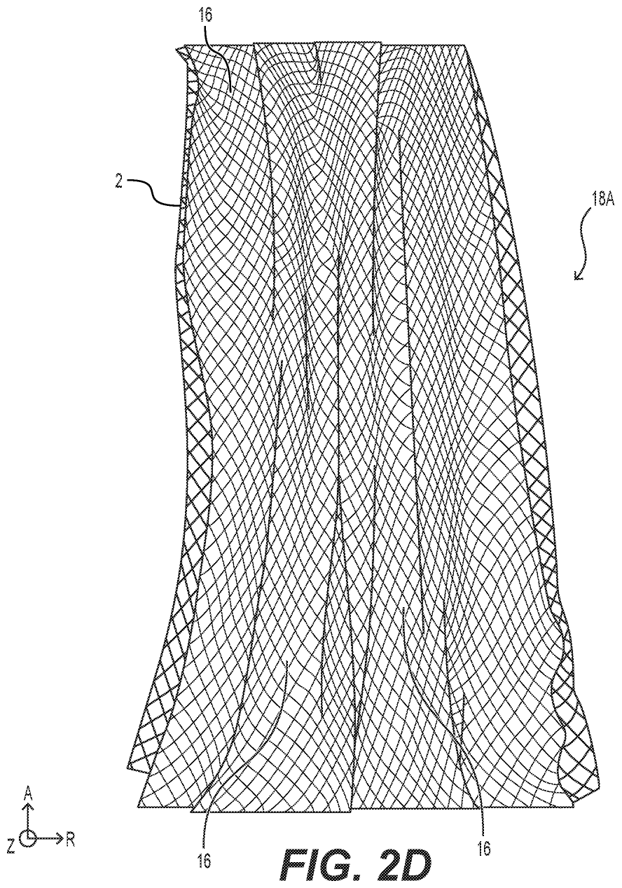

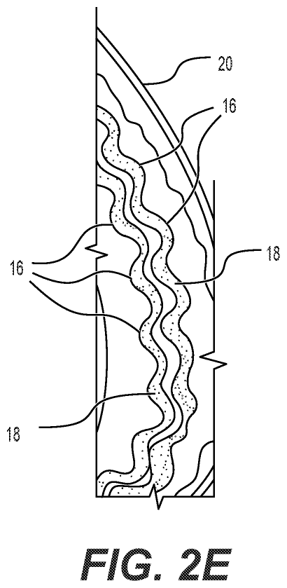

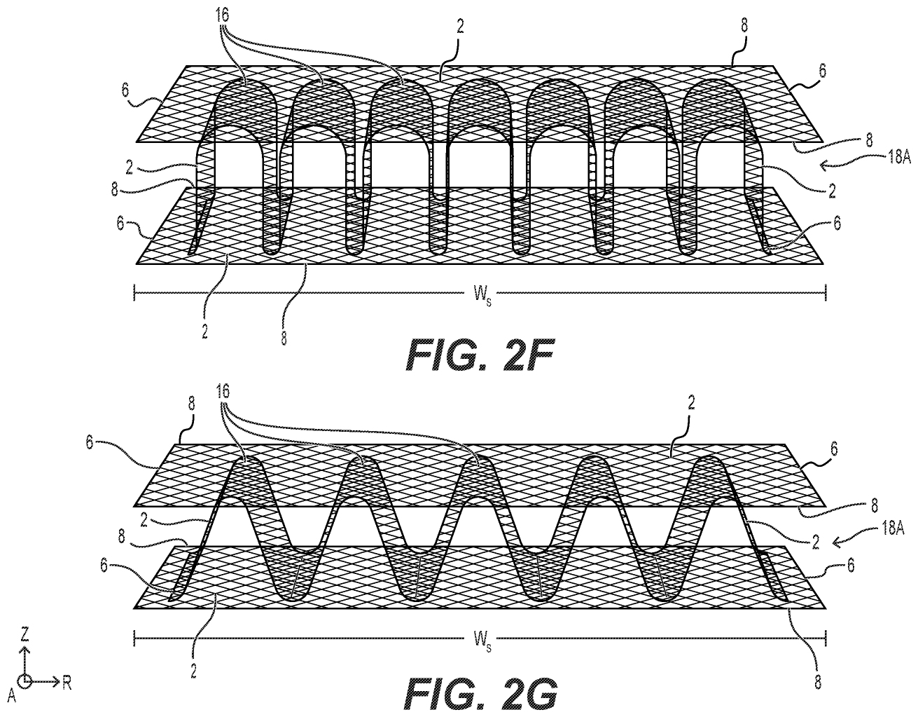

Mesh sheet 2 shown in FIGS. 1A-1B can be used either by itself or combined with one or more additional mesh sheets 2 to form a number of different three-dimensional mesh arrangements 18. One such arrangement is shown in FIGS. 2A-E. In particular, FIG. 2A shows a single mesh sheet 2 that has been manipulated to include pleats or corrugations 16. Corrugations 16 are formed by bringing together either lengthwise edges 6 of mesh sheet 2 (as shown in FIG. 2A) so that mesh sheet width W_{S} effectively becomes smaller as compared to flat mesh sheet 2 shown in FIGS. 1A-1B, or widthwise edges 8 of mesh sheet 2 so that mesh sheet length L_{S} effectively becomes smaller as compared to flat mesh sheet 2 shown in FIGS. 1A-1B. This “bunching,” “pleating,” or “corrugating” of mesh sheet 2 imparts mesh sheet 2 with three-dimensionality such that mesh sheet 2 becomes a three-dimensional mesh arrangement 18A. In particular, with corrugations 16, mesh sheet 2 extends in a non-negligible manner in a third direction Z perpendicular to both axial direction A and radial direction R. Adding corrugations 16 to mesh sheet 2 results in three-dimensional mesh arrangement 18A having a wave shape.

Mesh sheet 2 can be corrugated to a greater or lesser extent. For example, FIG. 2B shows mesh sheet 2 with seven corrugations 16, while FIG. 2C shows mesh sheet 2 with five corrugations 16. The effective mesh sheet width W_{S} of mesh sheet 2 in FIG. 2B is smaller than the effective mesh sheet width W_{S} of mesh sheet 2 in FIG. 2C because mesh sheet 2 in FIG. 2B is more corrugated than mesh sheet 2 shown in FIG. 2C.

While the number of corrugations 16 in mesh sheet 2 can vary, as discussed above, the shape of corrugations 16 when viewed along axial direction A (i.e., in cross-section) can also vary. For example, FIG. 2B shows corrugations 16 having more of a pill, capsule, or stadium shape, similar to a hemisphere placed on one short end of a rectangle, while FIG. 2C shows corrugations 16 having more of a rounded triangular shape. Any number of shapes of corrugations 16 is possible. Due to the memory characteristic of mesh sheet 2, corrugations 16 can also be expanded and retracted (i.e., mesh sheet width W_{S} can be varied). When corrugations 16 are expanded and more open, which decreases the mesh porosity of three-dimensional mesh arrangement 18A, they allow biological or synthetic agents to be more easily deposited onto mesh sheet 2. Corrugations 16 also help maintain such biological or synthetic agents in place once they have been deposited onto mesh sheet 2, which improves the overall likelihood that resulting three-dimensional mesh arrangement 18A will be accepted into the patient's body and provide sufficient support for the adjacent tissues therein. It is also possible to compress corrugations 16 to approximately their original, non-expanded position, which increases the mesh porosity of three-dimensional mesh arrangement 18A, and correspondingly increases the strength of three-dimensional mesh arrangement 18A and the support it provides to adjacent tissues once placed in the patient's body.

Furthermore, while FIGS. 2B-2C show corrugations 16 being more regular and precisely formed, corrugations 16 can also be imperfectly formed, as is the case with FIG. 2A, as corrugations 16 may be formed in mesh sheet 2 by hand (i.e., not by machine). FIG. 2D, which shows a top view of three-dimensional mesh arrangement 18A of FIG. 2A, also shows corrugations 16 formed in mesh sheet 2 by hand.

By corrugating mesh sheet 2 to create three-dimensional mesh arrangement 18A, additional volume is created as compared with mesh sheet 2 when flat and not corrugated (i.e., as shown in FIGS. 1A-1B). Transforming mesh sheet 2 into three-dimensional mesh arrangement 18A, then inserting one or more instances of the resulting three-dimensional mesh arrangement 18A into a cavity within the patient's body provides enhanced structural support to adjacent tissues due to the increased volume of three-dimensional mesh arrangement 18A as compared to flat mesh sheet 2, all while maintaining the benefits of absorbable mesh. As an example, FIG. 2E shows a side, cross-sectional view of two instances of three-dimensional mesh arrangement 18A, in its corrugated form, inserted into a cavity within patient's body 20, namely the breast. As shown, corrugations 16 of three-dimensional mesh arrangements 18A extend laterally with respect to patient's body 20. In this manner, three-dimensional mesh arrangements 18A can be used to provide support to three-dimensional cavities created within patient's body 20 (e.g., cavities created as a result of a lumpectomy).

In an embodiment, three-dimensional mesh arrangement 18A from FIGS. 2B-2C can be stacked with a second mesh sheet 2 and/or a third mesh sheet 2, as shown FIGS. 2F-2G, respectively. For example, second mesh sheet 2 could be disposed above corrugations 16 such that second mesh sheet 2 contacts corrugations 16 of three-dimensional mesh arrangement 18A at least in part. Third mesh sheet 2 could be disposed below corrugations 16 of three-dimensional mesh arrangement 18A such that third mesh sheet 2 contacts corrugations 16 at least in part. In this manner, second mesh sheet 2 and third mesh sheet 2 sandwich three-dimensional mesh arrangement 18A therebetween. Second and third mesh sheets 2 could be co-extensive with three-dimensional mesh arrangement 18A. Second and third mesh sheets 2 could also be co-planar with three-dimensional mesh arrangement 18A.

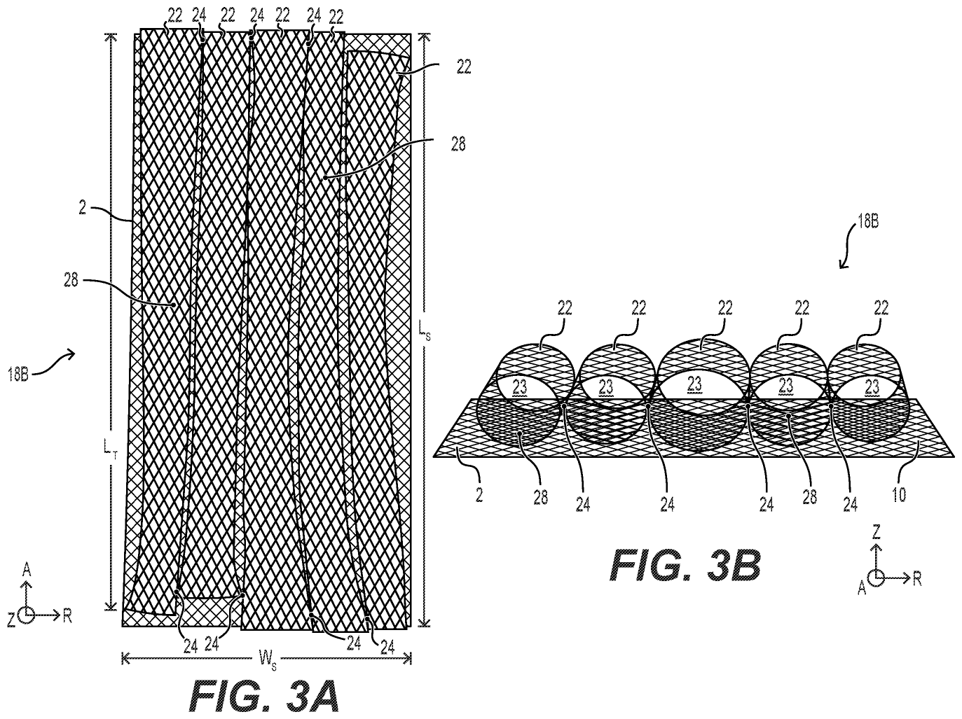

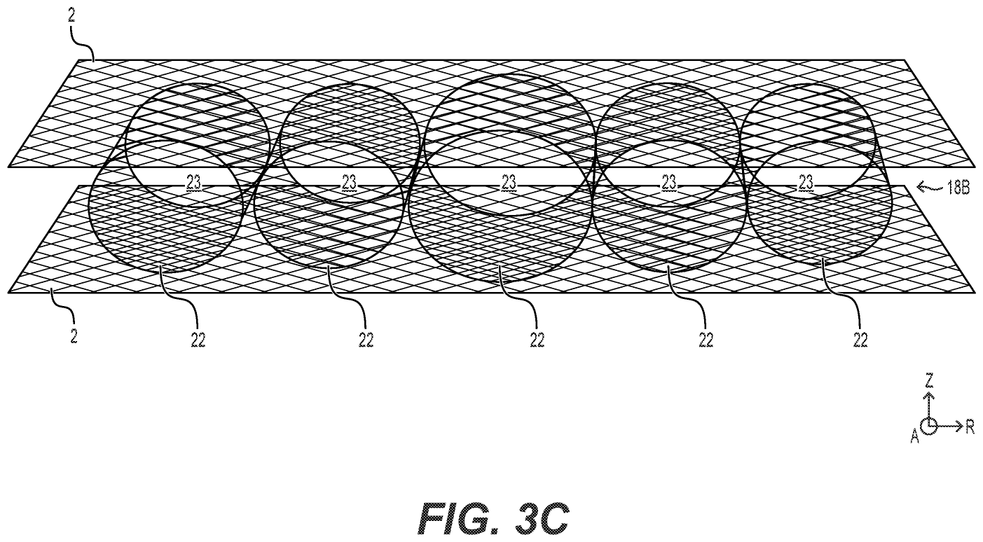

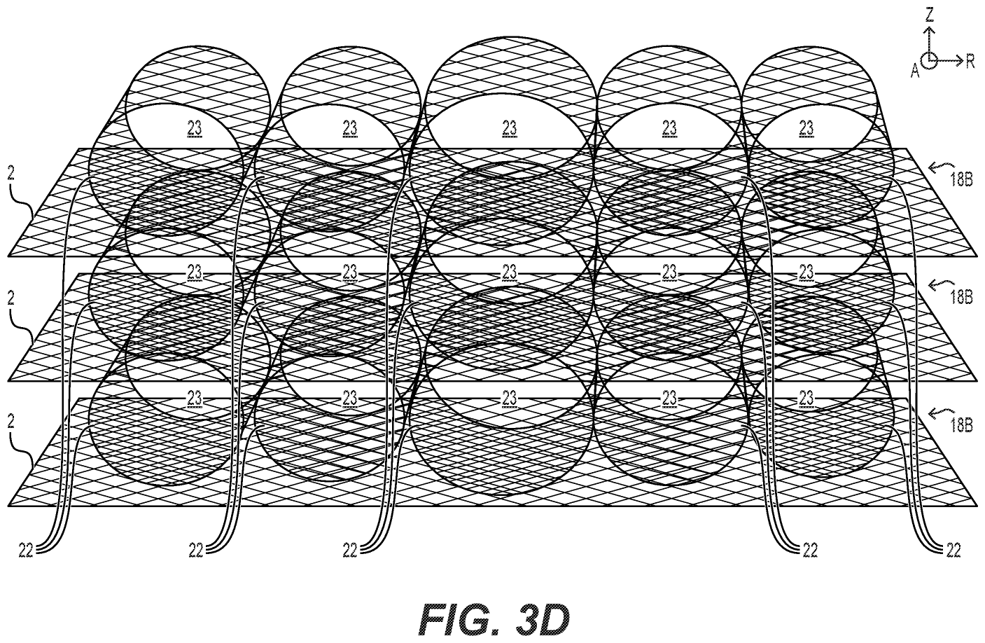

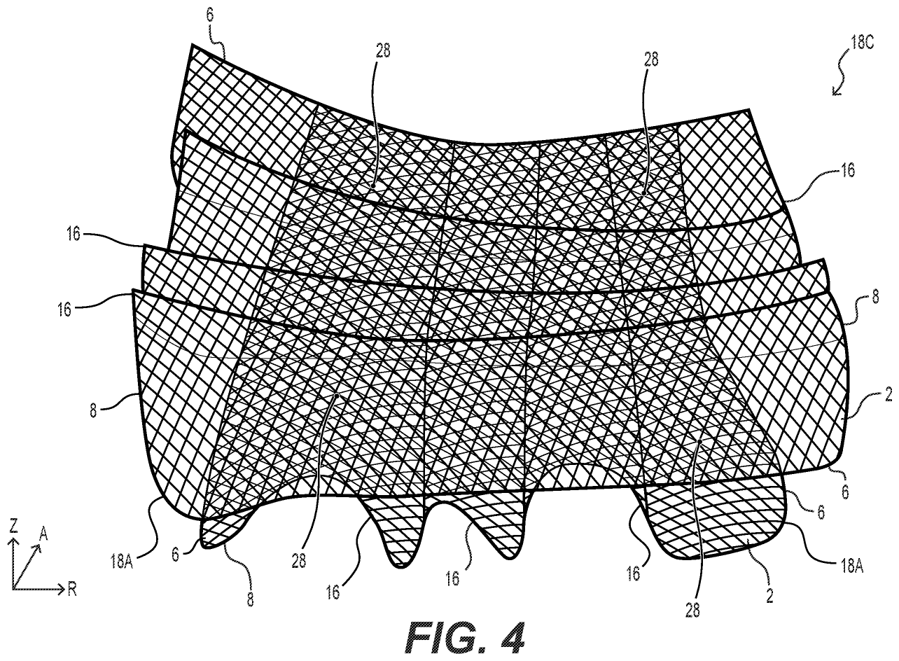

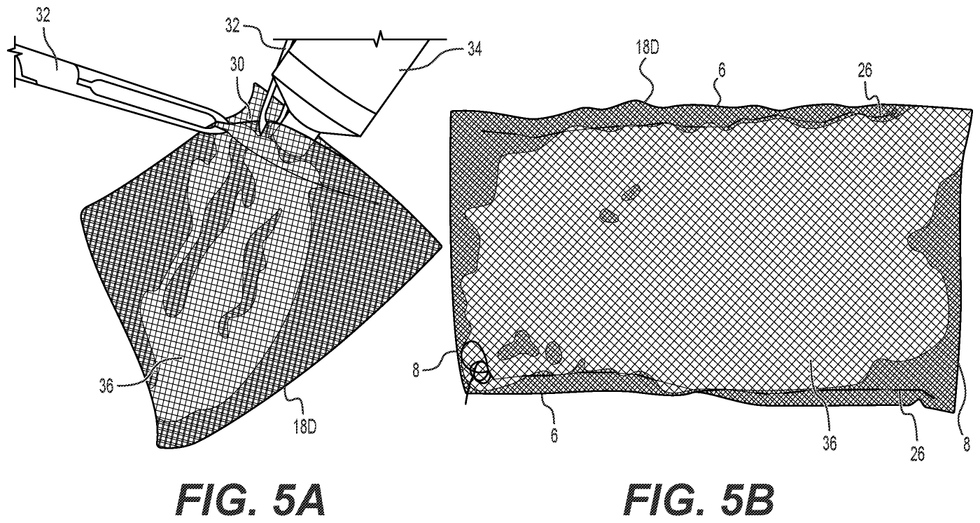

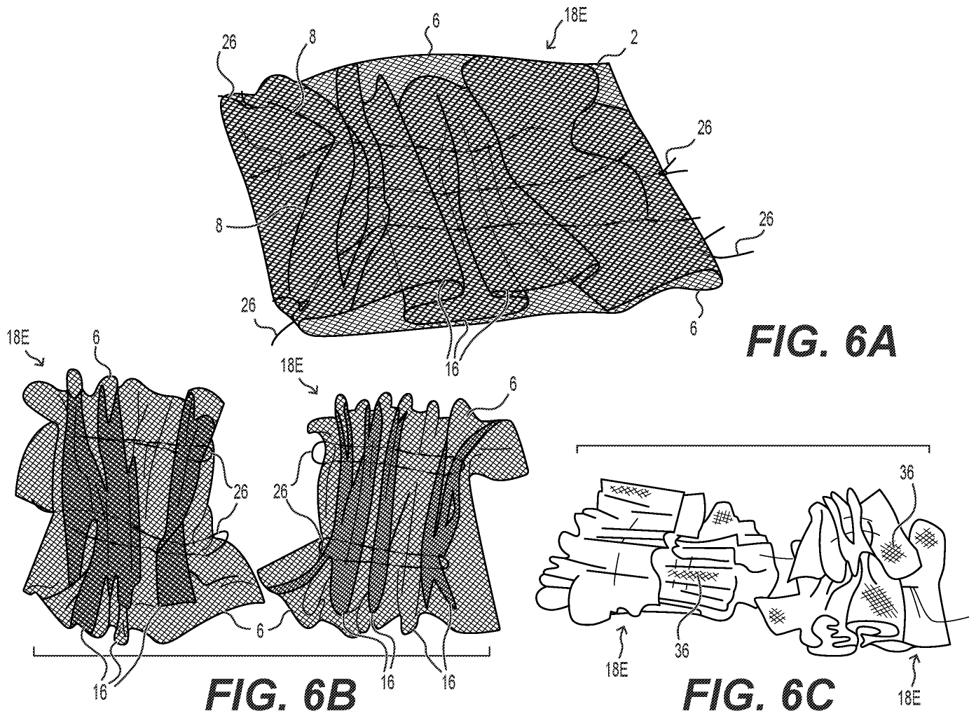

FIGS. 3A-3B show another embodiment of a three-dimensional mesh arrangement 18B. This three-dimensional mesh arrangement 18B includes a plurality of mesh tubes 22 disposed on a flat mesh sheet 2. Each mesh tube 22 may be formed by rolling a flat mesh sheet 2, such as flat mesh sheet 2 shown in FIGS. 1A-1B, or a portion thereof, such that mesh sheet 2's lengthwise edges 6 come into contact with one another. Lengthwise edges 6 are then joined together, at least partially, to form mesh tube 22. Lengthwise edges 6 can be joined together by a connecting thread, one or more sutures, adhesive, a melted polymer, or by melting sheet 2 along at least a part of lengthwise edges 6. Alternatively, widthwise edges 8 could be joined together instead of lengthwise edges 6. In either case, once rolled, each mesh tube 22 has a mesh tube length L_{T} along axial direction A. In an embodiment, each mesh tube length L_{T} is approximately equal, although the mesh tube length of each individual mesh tube 22 can vary. Rather than joining opposing edges of mesh sheet 2 together to form mesh tube 22, mesh tubes 22 could instead be formed using a braiding process so as to ensure that the diameter and porosity of each mesh tube 22 is maintained, and to provide a radial wall force to reduce compression or deformation of mesh tubes 22.

Mesh tubes 22 can have various cross-sectional shapes. For example, each mesh tube 22 could have a generally circular cross-section along its mesh tube length L_{T}. Each mesh tube 22 could instead have a generally oval cross-section along its mesh tube length L_{T}, as shown in FIG. 3B. Other cross-sectional shapes are possible. Each mesh tube 22 could have along its mesh tube length L_{T} a pill-, capsule-, or stadium-shaped cross section, similar to a hemisphere placed on one short end of a rectangle. Mesh tubes 22 could also have cross-sectional shapes different from one another or different from their adjacent mesh tubes 22. in any case, however, when mesh sheet 2 is rolled to form mesh tube 22, the resulting mesh tube 22 encloses a mesh tube volume 23. Each mesh tube volume 23 helps to impart three-dimensional mesh arrangement 18B with three-dimensionality so that three-dimensional mesh arrangement 18B can support and strengthen surrounding tissues within patient's body 20.

Plurality of mesh tubes 22 can be arranged in parallel on mesh sheet 2, extending along axial direction A. In an embodiment, each mesh tube 22 is affixed to at least one adjacent (i.e., in radial direction R) mesh tube 22 at at least one respective connection point 24. Each connection point 24 can comprise a connecting thread (e.g., such as connecting thread 26 shown in FIG. 5B), one or more sutures, adhesive, a melted polymer, or the melting together of two adjacent mesh tubes 22. In an embodiment, a connecting thread (e.g., such as connecting thread 26 shown in FIG. 5B) can extend through multiple connection points 24 so as to secure one or more mesh tubes 22 to one another.

In an embodiment, plurality of mesh tubes 22 is affixed to the first surface 10 of mesh sheet 2 at at least one anchor point 28. Like connection points 24, each anchor point 28 can comprise a connecting thread (e.g., such as connecting thread 26 shown in FIG. 5B), one or more sutures, adhesive, or a melted polymer. Each anchor point 28 can also be formed by the melting together of a point on a mesh tube 22 and a corresponding point on mesh sheet 2.

In an embodiment, each mesh tube length L_{T} can be approximately equal to mesh sheet length L_{S} of mesh sheet 2. Furthermore, plurality of mesh tubes 22 can include enough mesh tubes 22 that mesh tubes 22 extend over the entire width of mesh sheet width W_{S}. In this manner, plurality of mesh tubes 22 can be generally co-extensive with mesh sheet 2 in axial direction A and radial direction R. For example, as shown in FIGS. 3A-3B, plurality of mesh tubes 22 includes five mesh tubes 22, and plurality of mesh tubes 22 is generally co-extensive with mesh sheet 2. Plurality of mesh tubes 22 can also be co-planar with first surface 10 of mesh sheet 2.

In an embodiment, three-dimensional mesh arrangement 18B from FIGS. 3A-3B can include a second mesh sheet 2, as shown FIG. 3C. For example, second mesh sheet 2 could be disposed on plurality of mesh tubes 22 such that second mesh sheet 2 contacts plurality of mesh tubes 22 at least in part. In this manner, first mesh sheet 2 and second mesh sheet 2 sandwich mesh tubes 22 therebetween. Second mesh sheet 2 could be identical to first mesh sheet 2 or comparably sized to first mesh sheet 2. Second mesh sheet 2 could be co-extensive with first mesh sheet 2 and plurality of mesh tubes 22. Second mesh sheet 2 could also be co-planar with first mesh sheet 2 and plurality of mesh tubes 22.

In an embodiment, multiple instances of three-dimensional mesh arrangement 18B from FIGS. 3A-3B can be stacked on top one another, as shown FIG. 3D. For example, mesh sheet 2 of a second instance of three-dimensional mesh arrangement 18B could be disposed on plurality of mesh tubes 22 of a first instance of three-dimensional mesh arrangement 18B, while mesh sheet 2 of a third instance of three-dimensional mesh arrangement 18B could be disposed on plurality of mesh tubes 22 of the second instance of three-dimensional mesh arrangement 18B. In this manner, the three-dimensional mesh arrangement 18 shown in FIG. 31) includes three instances of three-dimensional mesh arrangement 18B from FIGS. 3A-3B. Second and third mesh sheets 2 could be identical to first mesh sheet 2 or comparably sized to first mesh sheet 2. Second and third mesh sheets 2 could be co-extensive with first mesh sheet 2 and its plurality of mesh tubes 22. Second and third mesh sheets 2 could also be co-planar with first mesh sheet 2 and its plurality of mesh tubes 22.

FIG. 4 shows another embodiment of a three-dimensional mesh arrangement 18C. This arrangement comprises two instances of three-dimensional mesh arrangement 18A of FIGS. 2A-E, one stacked atop another, such that at least one corrugation 16 of the plurality of corrugations of the top three-dimensional mesh arrangement 18A contacts at least one corrugation 16 of the plurality of corrugations of the bottom three-dimensional mesh arrangement 18A. The top three-dimensional mesh arrangement 18A is rotated with respect to the bottom three-dimensional mesh arrangement 18A. For example, the top three-dimensional mesh arrangement 18A could be rotated 90° with respect to the bottom three-dimensional mesh arrangement 18A. It is conceivable, however, that the top three-dimensional mesh arrangement 18A could be rotated by some angle other than 90° with respect to the bottom three-dimensional mesh arrangement 18A, In any case, however, the plurality of corrugations 16 of the top three-dimensional mesh arrangement 18A are transverse to the plurality of corrugations 16 of the bottom three-dimensional mesh arrangement 18A. The top and bottom three-dimensional mesh arrangements 18A in FIG. 4 could be essentially identical, or could vary from one another in one or more ways. For example, the number and/or shape of corrugations 16 of the top and bottom three-dimensional mesh arrangements 18A could be different. The overall dimensions of top and bottom three-dimensional mesh arrangements 18A, including mesh sheet length L_{S} and mesh sheet width W_{S}, could be different.

Top three-dimensional mesh arrangement 18A could also be attached to bottom three-dimensional mesh arrangement 18A at one or more anchor points 28, similar to how in FIGS. 3A-3B plurality of mesh tubes 22 may be connected to mesh sheet 2 at one or more anchor points 28. As before, each anchor point 28 can comprise a connecting thread (e.g., such as connecting thread 26 shown in FIG. 5B), one or more sutures, adhesive, or a melted polymer. Each anchor point 28 can also be formed by the melting together of a point on top three-dimensional mesh arrangement 18A and a corresponding point on bottom three-dimensional mesh arrangement 18A.

By stacking one instance of three-dimensional mesh arrangement 18A on top of another instance of three-dimensional mesh arrangement ISA, the resulting three-dimensional mesh arrangement 18C can occupy a greater volume within patient's body 20 than one instance of three-dimensional mesh arrangement 18A alone. Furthermore, by rotating top three-dimensional mesh arrangement 18A with respect to bottom three-dimensional mesh arrangement 18A, corrugations 16 of each three-dimensional mesh arrangement 18A are transverse to one another, resulting in an overall three-dimensional mesh arrangement 18C that is capable of providing more support than just one instance of three-dimensional mesh arrangement 18A by itself.

FIGS. 5A-B show yet another embodiment of a three-dimensional mesh arrangement 18D. This arrangement comprises two instances of mesh sheet 2 of FIGS. 1A-1B stacked atop one another then connected so as to form a bag. In the embodiment shown, one or more connecting threads 26 are used to connect the corresponding lengthwise edges 6 of each mesh sheet to one another (e.g., by sewing) in order to form a pocket 30 therebetween. The two instances of mesh sheet 2 in three-dimensional mesh arrangement 18D can be connected to one another in other ways. For example, corresponding lengthwise edges 6 of each mesh sheet 2 can be joined together by one or more sutures, adhesive, a melted polymer, or by melting each mesh sheet 2 along at least a part of lengthwise edges 6. The two instances of mesh sheet 2 also can, alternatively or in addition to the connection along lengthwise edges 6, be at least partially connected along widthwise edges 8 so as to form pocket 30. The size of three-dimensional mesh arrangement 18D can also be varied by changing the size of mesh sheets 2 used to form three-dimensional mesh arrangement 18D.

Pocket 30 can receive a material. For example, using forceps 32, the two instances of mesh sheet 2 of three-dimensional mesh arrangement 18D can be pulled apart from one another, as shown in FIG. 5A, so as to provide access to pocket 30. Syringe 34, which contains biological or synthetic agent 36, can then be inserted into pocket 30 (i.e., between the two instances of mesh sheet 2) so as to inject biological or synthetic agent 36 into pocket 30. The pocket design of three-dimensional mesh arrangement 18D helps to retain biological or synthetic agent 36 therein, increasing the overall likelihood that three-dimensional mesh arrangement 18D will be accepted into the patient's body 20 and provide sufficient support for adjacent tissues. It is also possible that two or more instances of three-dimensional mesh arrangement 18D, with or without biological or synthetic agent 36 therein, can be layered to create various shapes to fill defects or cavities within the patient's body 20 (e.g., following removal of cancerous tissue in a lumpectomy or even a mastectomy).

FIGS. 6A-6C show a further embodiment of a three-dimensional mesh arrangement 18E. In this arrangement, widthwise edges 8 of mesh sheet 2 are brought together and one or more connecting threads 26 are secured around the periphery of mesh sheet 2, then tied off to secure together widthwise edges 8. Adjusting the overall effective length of the one or more connecting threads 26 can change the number of corrugations 16 in three-dimensional mesh arrangement 18E. For example, by cinching the one or more connecting threads 26, the number of corrugations 16 in three-dimensional mesh arrangement 18E can be increased. Conversely, by loosening the one or more connecting threads 26, the number of corrugations 16 in three-dimensional mesh arrangement 18E can be decreased. In this manner, a surgeon manipulating three-dimensional mesh arrangement 18E can alter the number of corrugations 16 therein as desired.

FIG. 6B shows two instances of three-dimensional mesh arrangement 18E. As shown in FIG. 6B, two or more instances of three-dimensional mesh arrangement 18E can be used to fill a cavity within patient's body 20. FIG. 6C shows two instances of three-dimensional mesh arrangement 18E that have been coated in biological or synthetic agent 36 (e.g., human fat). Due to the presence of corrugations 16, biological or synthetic agent 36 is better retained on three-dimensional mesh arrangements 18E, increasing the likelihood that the arrangements will be accepted within patient's body 20 and provide the required tissue support.

In addition to the embodiments described and shown herein, it is also contemplated that any combination of three-dimensional mesh arrangements 18A-18E could be combined, with or without instances of mesh sheet 2 disposed therebetween, on top, or below, to create other three-dimensional mesh arrangements 18. For example, three-dimensional mesh arrangement 18A could be disposed between two instances of mesh sheet 2 to form another three-dimensional arrangement 18. In another example, an instance of three-dimensional mesh arrangement 18A could be stacked on an instance of three-dimensional mesh arrangement 18B, with or without a mesh sheet 2 therebetween. Moreover, two or more instances of each three-dimensional mesh arrangement 18A-18E could be combined, with or without instances of mesh sheet 2 disposed therebetween, on top, or below, to create other three-dimensional mesh arrangements 18. As one example, two instances of three-dimensional mesh arrangement 18C could be stacked one atop another, with or without a mesh sheet 2 therebetween.

Various other aspects are contemplated herein, several of which are set forth in the paragraphs below. It is explicitly contemplated that any aspect or portion thereof can be combined, with or without the embodiments described herein, to form an aspect.

Aspect 1: A surgical mesh that is corrugated either with a W pattern (pleated) or sinusoidal pattern.

Aspect 2: A surgical mesh that is corrugated into cylindrical layers.

Aspect 3: The mesh can be stretched, thus opening the cylinders and then contract to re-approximate.

Aspect 4: Pre-formed tubular constructs can be layered together or placed between two layers of mesh forming a non-expandable structural scaffold.

Aspect 5: The mesh is heat molded in order to achieve the desired shape.

Aspect 6: The mesh can be layered to achieve three-dimensional shapes.

Aspect 7: The tubular construct is more resistant to collapse and is better able to retain its projection. The mesh is formed into a bag into which the fat is injected. A three-dimensional structure is thus formed.

Aspect 8: The mesh has a microporous structure appropriate to retain applied material. Platelet-rich plasma is added to the mesh containing the biological material. The platelet-rich plasma is allowed to clot forming a gelatinous mass in the tubule thus forming the optimum scaffold for adjacent tissue to migrate into blood vessels, fat cells, and fibroblasts forming collagen.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of B or C” should be interpreted as including any singular entity from the listed elements, e.g., A, any subset from the listed elements, e.g., A and B, or the entire list of elements A, B and C.

The present disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### LIST OF REFERENCE SYMBOLS

2 mesh sheet
4 openings
6 lengthwise edge
8 widthwise edge
10 first surface
12 second surface
14 node
16 corrugations/pleats
18 three-dimensional mesh arrangement
20 patient's body
22 mesh tube
23 mesh tube volume
24 connection point
26 connecting thread.
28 anchor point
30 pocket
32 forceps
34 syringe
A axial direction
R radial direction
third direction
L_{S} mesh sheet length
W_{S} mesh sheet width
L_{T} mesh tube length

What is claimed is:

1. An arrangement, comprising:
a plurality of mesh tubes arranged in parallel and extending along an axial direction, each mesh tube of the plurality of mesh tubes having a mesh tube length in the axial direction; and
a first mesh sheet having a first surface,
wherein the plurality of mesh tubes is disposed on the first surface.

2. The arrangement of claim 1, wherein each mesh tube is affixed to at least one adjacent mesh tube at at least one respective connection point.

3. The arrangement of claim 2, further comprising:
at least one connecting thread passing through one or more of the connection points.

4. The arrangement of claim 2, wherein each connection point comprises at least one of melted polymer, adhesive, or a suture.

5. The arrangement of claim 1, wherein the plurality of mesh tubes is affixed to the first surface at at least one anchor point.

6. The arrangement of claim 5, wherein the at least one anchor point comprises at least one of melted polymer, an adhesive, a suture, or a connecting thread.

7. The arrangement of claim 1, wherein each mesh tube comprises a rolled mesh sheet.

8. The arrangement of claim 7, wherein, for each rolled mesh sheet, two edges thereof are joined together, at least partially, to form the mesh tube.

9. The arrangement of claim 1, wherein the first mesh sheet has an approximately rectangular shape having a first mesh sheet length extending in the axial direction and a first mesh sheet width extending in a radial direction.

10. The arrangement of claim 9, wherein each mesh tube length is approximately equal to the first mesh sheet length.

11. The arrangement of claim 1, each mesh tube length is approximately equal.

12. The arrangement of claim 1, wherein each mesh tube has one of a generally circular cross-section along the mesh tube length, a generally oval cross-section along the mesh tube length, or a pill-shaped cross-section along the mesh tube length.

13. The arrangement of claim 1, wherein the plurality of mesh tubes is co-planar with the first surface.

14. The arrangement of claim 1, further comprising:
a second mesh sheet having a second surface which is disposed on the plurality of mesh tubes such that the second surface contacts the plurality of mesh tubes at least in part.

15. The arrangement of claim 14, wherein the second surface is co-planar with the first surface.

16. An arrangement, comprising:
a first mesh sheet comprising a plurality of corrugations; and
a second mesh sheet comprising a plurality of corrugations,
wherein the second mesh sheet is disposed on the first mesh sheet such that at least one corrugation of the plurality of corrugations of the second mesh sheet contacts at least one corrugation of the plurality of corrugations of the first mesh sheet, and
wherein the plurality of corrugations of the second mesh sheet are transverse to the plurality of corrugations of the first mesh sheet.

17. An arrangement, comprising:
a first rectangular mesh sheet having a mesh sheet length, a mesh sheet width, a first lengthwise edge, and a second lengthwise edge;
a second rectangular mesh sheet having a mesh sheet length, a mesh sheet width, a first lengthwise edge, and a second lengthwise edge, the mesh sheet length and mesh sheet width of the second rectangular mesh sheet being approximately equal to the mesh sheet length and mesh sheet width, respectively, of the first rectangular mesh sheet, the second mesh sheet being disposed on the first mesh sheet so as to be generally coextensive with the first mesh sheet;
a first connecting thread connecting the first lengthwise edge of the first rectangular mesh sheet and the first lengthwise edge of the second rectangular mesh sheet; and
a second connecting thread connecting the second lengthwise edge of the first rectangular mesh sheet and the second lengthwise edge of the second rectangular mesh sheet so as to form a pocket between the first rectangular mesh sheet and the second rectangular mesh sheet.
